(19) United States
(12) Patent Application Publication
(10) Pub. No.: US 2009/0233990 A1
(43) Pub. Date: Sep. 17, 2009

(54) GENERATION OF BIOLOGICAL PACEMAKER ACTIVITY

(76) Inventors: Hee Cheol Cho, Hanover, MD (US); Eduardo Marban, Beverly Hills, CA (US); Daniel Sigg, St. Paul, MN (US)

Correspondence Address: CAMPBELL NELSON WHIPPS, LLC, 408 ST. PETER STREET, SUITE 240, ST. PAUL, MN, 55102, US

(21) Appl. No.: 12/263,281

(22) Filed: Oct. 31, 2008

Related U.S. Application Data
(60) Provisional application No. 60/984,581, filed on Nov. 1, 2007.

(51) Int. Cl. A61K 31/7052; C12N 5/00; C12N 15/85; C12N 15/86; C12N 15/63; A61P 9/00

(52) U.S. Cl. 514/44 R; 435/375; 435/455; 435/456; 435/320.1; 435/325

(57) ABSTRACT

Compositions and methods for enhancing hyperpolarization-activated cation inward current and disrupting inwardly rectifying potassium current of cells are described. The compositions and methods may be employed to cause the cells to become biological pacemaker cells, e.g. to become more like SA node cells, and to undergo spontaneous oscillating action potentials.

### RELATED APPLICATION

This application claims the benefit of Provisional Application Ser. No. 60/984,581, filed on Nov. 1, 2007, which application is hereby incorporated herein by reference in its entirety to the extent that it does not conflict with the present disclosure.

### FIELD

The present disclosure relates to compositions, apparatuses, and methods for generating biological pacemaker function in cells, and more particularly to enhancing hyperpolarization-activated cation conductance and disrupting inward rectifying potassium conductance of non-pacemaker cells.

### BACKGROUND

The heart is a continuously beating organ, but it cannot do so on its own without the specialized pacemaker cells and tissue of the heart. The sinoatrial (SA) node is the primary endogenous pacemaker of the heart and has the ability to generate spontaneous beat as well, however, at significantly lower rates. Recently, use of an ion channel over-expression has been examined to induce biological pacemaker activity in the heart. One such approach tries to unleash the cardiomyocytes' innate ability of spontaneous contraction. This is done by over-expressing a dominant negative mutant of an inward rectifier ion channel, Kir2.1AAA, which suppresses the ability of myocytes to clamp the membrane potential at rest. Without the resistance to remain at rest, the myocytes undergo cyclical undulations of its membrane potential, behaving as a pacemaker. The other strategy tries to up-regulate pacemaker ionic currents by pacemaker (hyperpolarization-activated cyclic nucleotide “HCN”) channel overexpression. Over-expression of HCN channels in non-pacemaker myocytes has been shown to win over the tendency of the myocytes to remain at rest and induce pacemaker activity by increasing inward cation current during hyperpolarization. However, such approaches have not been shown to maintain their function as a biological pacemaker. The frequency of observing in vivo pacemaker activity (i.e., efficiency) and the duration of such activity (i.e. consistency) are less than ideal and call for an improved strategy.

### BRIEF SUMMARY

The present disclosure describes compositions of matter and methods for achieving both disruption of inwardly rectifying potassium currents and enhancement of hyperpolarization-activated cation conductance.

In an embodiment, a method is described. The method includes (i) expressing an exogenous dominant negative Kir2.1 mutant inwardly rectifying potassium channel in a cell and (ii) expressing an exogenous hyperpolarization-activated cation (HCN) channel in the cell. The expression of the dominant negative Kir2.1 mutant and the HCN channel results in spontaneous oscillating action potentials in the cell.

In an embodiment, a method for inducing spontaneous oscillating action potentials in cardiomyocytes is described. The method includes expressing a Kir2.1ER mutant in the cardiomyocyte.

In an embodiment, a method is described. The method includes (i) identifying a cell that endogenously expresses an inwardly rectifying potassium channel and (ii) introducing into the cell a genetic construct comprising a polynucleotide. The polynucleotide, when expressed by the cell, disrupts the inwardly rectifying potassium current and increases inward hyperpolarization activated cation current.

### BRIEF DESCRIPTION OF THE DRAWINGS

FIG. 1A is the amino acid sequence of human HCN1 (SEQ. ID. NO. 1), according to Genbank Accession No. NMO21072.

FIG. 1B is the amino acid sequence of a truncated HCN1 (SEQ. ID. NO. 8)

FIG. 2 is the amino acid sequence of human HCN2 (SEQ. ID. NO. 2), according to Genbank Accession No. NM01194.

FIG. 3 is the amino acid sequence of human HCN3 (SEQ. ID. NO. 3), according to Genbank Accession No. NM020897.

FIG. 4A is the amino acid sequence of human HCN4 (SEQ. ID. NO. 4), according to Genbank Accession No. NM005477.

FIG. 4B is the amino acid sequence of a truncated HCN4 (SEQ. ID. NO. 9)

FIG. 5A is the amino acid sequence of human Kir2.1 (SEQ. ID. NO. 5), according to Genbank Accession No. AS150819.′

FIGS. 5B-C are amino acid sequences of mutant human Kir2.1; namely, Kir2.1AAA (SEQ. ID. NO. 6) (5B) and Kir2.1ER (SEQ ID. NO. 7) (5C).

FIG. 6 is a schematic diagram of a right side of a heart having an anterior-lateral wall peeled back.

FIG. 7 is a schematic diagram of the right side of a heart similar to that shown in FIG. 6

FIG. 8 is a current vs. voltage graph obtained by co-expressing wild-type and Kir2.1ER channels by Ad-Kir2.1ER-IRES-Kir2.1WT in HEK293 cells with and without 1 mM BaCl₂.

FIGS. 9A-B are voltage graphs over time of cardiomyocytes in vivo transduced with Ad-Kir2.1ER-IRES-GFP (9A) and control myocytes transduced with GFP alone (9B).

FIGS. 10A-B are electrocardiogram recordings three days after Ad-Kir2.1ER-IRES-GFP injection into the apex of a guinea pig heart (10A) and control (10B).

FIG. 11 is a vector map of a construct containing DNA encoding a truncated HCN1 channel and a Kir2.1 AAA channel.

FIG. 12 is a vector map of a construct containing DNA encoding a truncated HCN1 channel and a Kir2.1 AAA channel.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

The terms “encodes”, “encoding”, “coding sequence”, and similar terms as used herein, refer to a nucleic acid sequence that is transcribed (in the case of DNA) and translated (in the case of mRNA) into a polypeptide in vitro or in vivo when placed under control of appropriate regulatory sequences.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

The present disclosure relates to compositions and methods for enhancing hyperpolarization-activated cation inward current and disrupting inwardly rectifying potassium current of cells. The compositions and methods may be employed to cause the cells to become biological pacemaker cells, e.g. to become more like SA node cells, and to undergo spontaneous oscillating action potentials. In various embodiments, the cells may also be paced with a pacemaker device. The cells may be in vivo, ex vivo, or in vitro. When employed in vivo, the compositions and methods may, in various embodiments, be for treating conditions of a patient associated with abnormal or dysfunctional pacemaker cells. For example, the compositions or methods described herein may be employed to treat conditions associated with abnormal or dysfunctional cardiac conduction system, such as SAN disease, sick sinus syndrome, SAN block, AVN block, and other brady-cardia syndromes such as drug-induced bradycardia, vagal induced syncopies. Of course, the compositions and methods may be employed for investigatory purposes; e.g. to study the function of cells, or the like, when exposed to the compositions or employed in the methods. Such studies may ultimately lead to therapeutic applications.

Any suitable mechanism for enhancing hyperpolarization-activated cation inward current and disrupting inwardly rectifying potassium current may be employed. In various embodiments, hyperpolarization-activated cation inward current is enhanced by overexpressing an HCN channel in a cell and inwardly rectifying potassium current is disrupted by overexpressing a dominant negative Kir mutant in the cell.

Suitable polynucleotides for use with the vectors and methods described herein can be obtained from a variety of public sources including, without limitation, GenBank (National Center for Biotechnology Information (NCBI)), EMBL data library, SWISS-PROT (University of Geneva, Switzerland), the PIR-International database; and the American Type Culture Collection (ATCC)(10801 University Boulevard, Manassas, Va. 20110-2209). See generally, Benson, D. A. et al, Nucl. Acids. Res., 25:1 (1997) for a description of GenBank. The particular polynucleotides useful with the present invention are readily obtained by accessing public information from GenBank.

Any functional exogenous HCN channel may be expressed in a cell and employed according to the teachings presented herein. Functional HCN channels include full-length wild-type channels and functional variants or fragments thereof. While it is not necessary for the HCN channel to be from the same species as the cell in which it is exogenously expressed, it may be desirable for the HCN channel and the cell to be of similar origin. For example, it may be desirable for the cell and the HCN channel to be of mammalian origin. In various embodiments, the cell and the HCN channel are both of human origin.

To date, four isoforms of the HCN channel have been identified; namely, HCN1, HCN2, HCN3, and HCN4. Any one or more of such HCN channels may be employed according to the teachings provided herein. Examples of suitable HCN channels that may be employed can be found on Genbank, and include: NM001194 (Homo sapiens hyperpolarization activated cyclic nucleotide-gated potassium channel 2 (HCN2), mRNA); NM021658 (Rattus norvegicus hyperpolarization-activated, cyclic nucleotide-gated K+4 (Hcn4), mRNA); NM053685 (Rattus norvegicus hyperpolarization-activated cyclic nucleotide-gated potassium channel 3 (Hcn3), mRNA); NM008227 (Mus musculus hyperpolarization-activated, cyclic nucleotide-gated K+3 (Hcn3), mRNA); NM005477 (Homo sapiens hyperpolarization activated cyclic nucleotide-gated potassium channel 4 (HCN4), mRNA); NM001081192 (Mus musculus hyperpolarization-activated, cyclic nucleotide-gated K+4 (Hcn4), mRNA); NM021072 (Homo sapiens hyperpolarization activated cyclic nucleotide-gated potassium channel 1 (HCN1), mRNA); NM053684 (Rattus norvegicus hyperpolarization activated cyclic nucleotide-gated potassium channel 2 (Hcn2), mRNA); NM020897 (Homo sapiens hyperpolarization activated cyclic nucleotide-gated potassium channel 3 (HCN3), mRNA); NM010408 (Mus musculus hyperpolarization-activated, cyclic nucleotide-gated K+1 (Hcn1), mRNA); NM008226 (Mus musculus hyperpolarization-activated, cyclic nucleotide-gated K+2 (Hcn2), mRNA); NM053375 (Rattus norvegicus hyperpolarization-activated cyclic nucleotide-gated potassium channel 1 (Hcn1), mRNA); AY686751 (Canis familiaris cardiac hyperpolarization-activated current subunit 2 (HCN2) mRNA, partial cds); AY686750 (Canis familiaris cardiac hyperpolarization-activated current subunit 4 (HCN4) mRNA, partial cds); AF421883 (Oncorhynchus mykiss hyperpolarization-activated cyclic nucleotide-gated cation channel 1 (HCN1) mRNA, complete cds); AF155170 (Oryctolagus cuniculus hyperpolarization-activated cation channel 4 (HCN4) mRNA, partial cds); AF155169 (Oryctolagus cuniculus hyperpolarization-activated cation channel 3 (HCN3) mRNA, partial cds); AF155168 (Oryctolagus cuniculus hyperpolarization-activated cation channel 2 (HCN2) mRNA, partial cds); AF155167 (Oryctolagus cuniculus hyperpolarization-activated cation channel 1 (HCN1) mRNA, partial cds); AF155166 (Rattus norvegicus hyperpolarization-activated cation channel 4 (HCN4) mRNA, partial cds); AF155165 (Rattus norvegicus hyperpolarization-activated cation channel 3 (HCN3) mRNA, partial cds); AF155164 (Rattus norvegicus hyperpolarization-activated cation channel 2 (HCN2) mRNA, partial cds); and AF155163 (Rattus norvegicus hyperpolarization-activated cation channel 1 (HCN1) mRNA, partial cds), with Genbank Accession Number and Genbank description following in parentheses. One of skill in the art will understand that mRNA sequence and amino acid sequence for the above may be readily obtained via Genbank or other similar database.

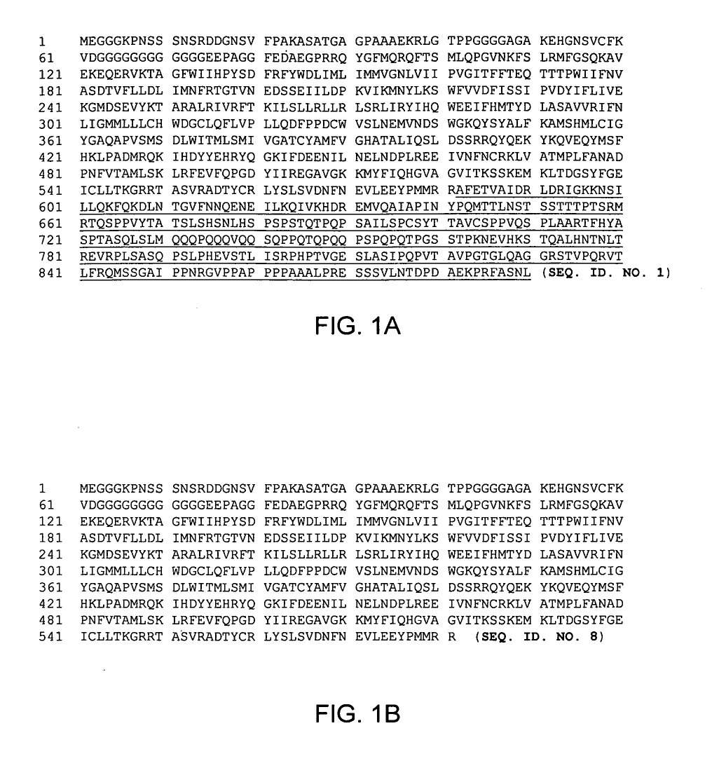

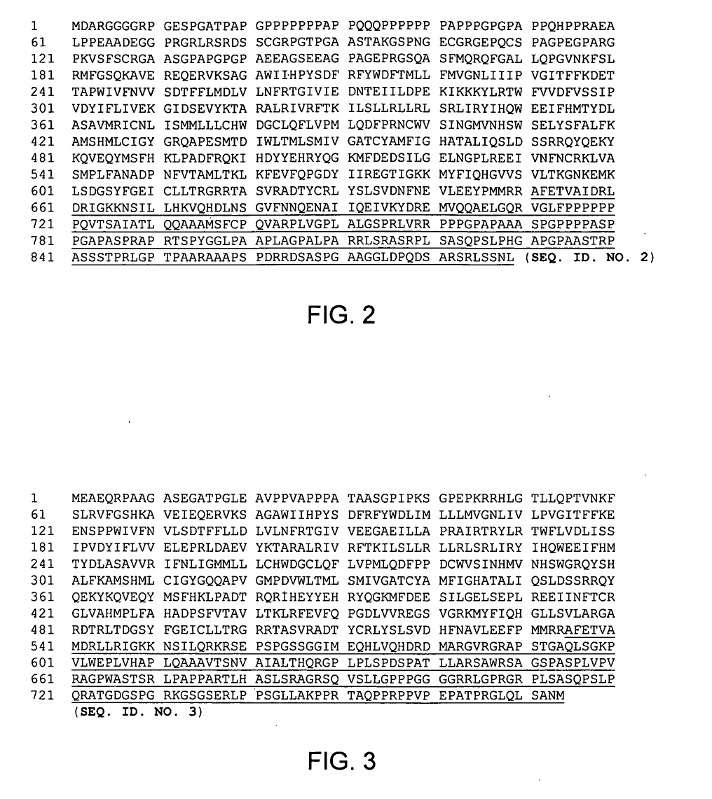

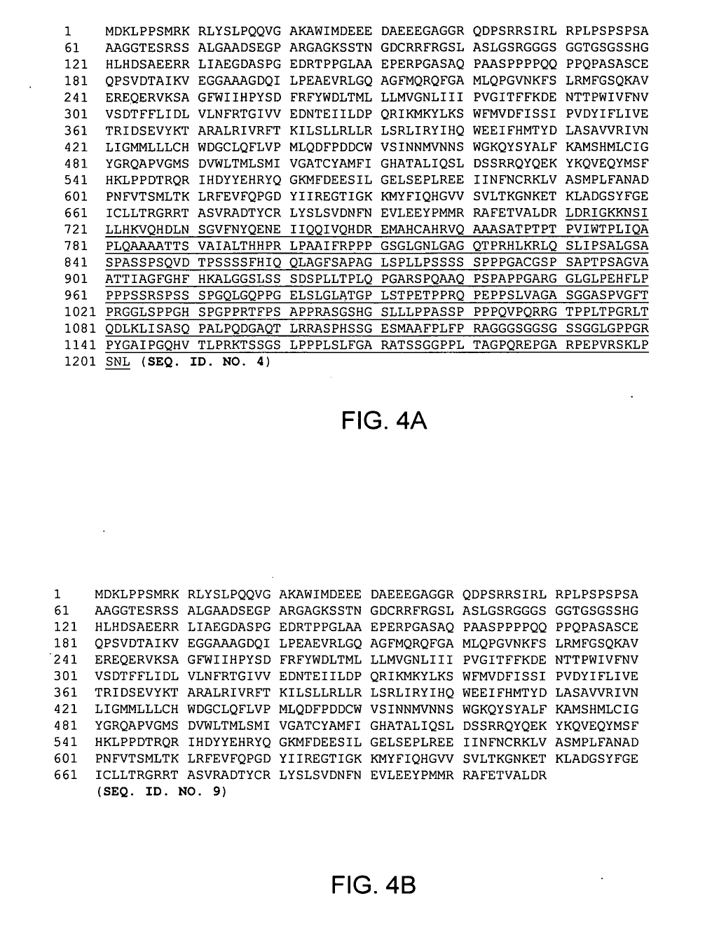

In various embodiments, a C-terminal end, or portion thereof, of an HCN channel is truncated at a position following the cyclic nucleotide binding site. Truncation may allow for improved packaging into viral vectors. By way of example, human HCN1 (SEQ. ID. NO. 1) can be truncated anywhere from amino acid 582-890 (see FIG. 1A for amino acid sequence, with truncatable amino acids underlined); human HCN2 (SEQ. ID. NO. 2) can be truncated anywhere from amino acid 651-889 (see FIG. 2 for amino acid sequence, with truncatable amino acids underlined); human HCN3 (SEQ. ID. NO. 3) can be truncated anywhere from amino acid 535-744 (see FIG. 3 for amino acid sequence, with truncatable amino acids underlined); or human HCN4 (SEQ. ID. NO. 4) can be truncated anywhere from amino acid 711-1203 (see FIG. 4A for amino acid sequence, with truncatable amino acids underlined). Specific examples of truncated HCN1 (SEQ. ID. NO. 8) and truncated HCN4 (SEQ. ID. NO. 9) are shown in FIG. 1B and FIG. 4B, respectively.

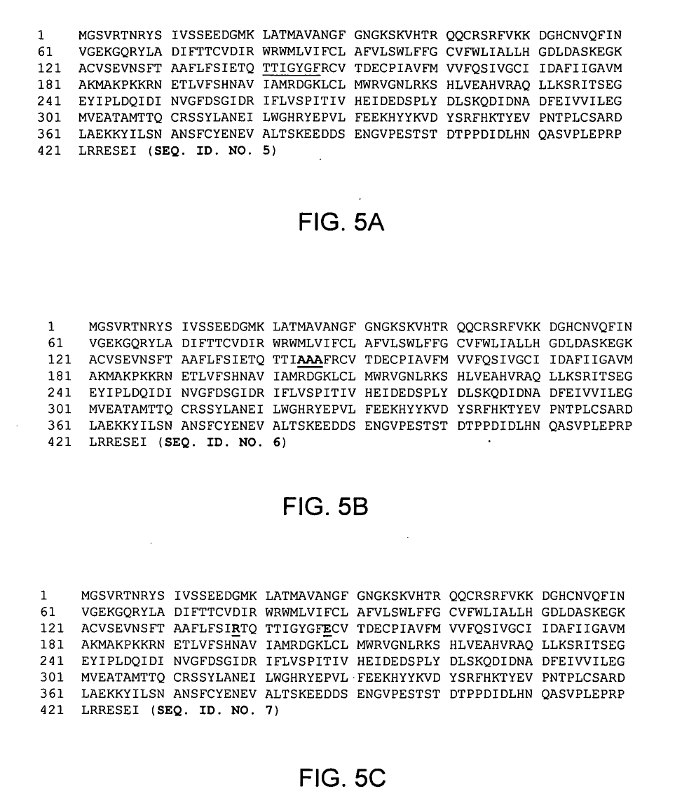

Any dominant negative Kir2.1 channel may be expressed in a cell and employed according to the teachings presented herein. A dominant negative Kir2.1 channel will suppress inwardly rectifying potassium current when expressed in conjunction with wild-type Kir2.1 (SEQ. ID. NO. 5). It is contemplated that any non-conservative amino acid substitution in the selectivity filter region (position 141, threonine, to position 147, phenylalanine) will be effective to generate a dominant negative Kir2.1 channel (see FIG. 5A for a wild-type sequence of human Kir2.1, with positions 141-147 underlined).

Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic: trp, tyr, phe. Non-conservative substitutions will entail exchanging a member of one of these classes for another class. Such substituted residues also may be introduced into the conservative substitution sites or, more preferably, into the remaining (non-conserved) sites. The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the Kir2.1 variant DNA.

A wild-type human Kir2.1 amino acid sequence (SEQ. ID. NO. 5), having Genbank Accession Number AS150819, is shown in FIG. 5A, which sequence may be modified to create a dominant negative Kir2.1 channel. Additional examples of Kir2.1 channels that may be modified may be found by searching Genbank, and include those associated with Genbank accession numbers: NM008425; NM015049; NM001364; NM174373; NM017296; NM000891; NM010603; BC152811; NM053981; NM021012; NM011807; NM170720; NM013348; NM152868; NM031602; NM019621; NM004981; DQ435677; DQ435676; DQ435675; DQ43567; NM001003120; EF570139; NM170742; NM170741; NM018658; BC127487; NW001464511; XM001195958; NW001333837; XM792080; NW926918; NT010641; XM965464; NW001093987; AM158252; NW876331; AY052548; AF187872; AF153819; AF153818; AH009400; AF153814; AF153813; AF153812; AF153811; AF153810; AF153809; AH009401; AF153820; AF153817; AF153816; AF153815; AF183915; AF183914; AF277647; U95369; and AF021142.

In various embodiments, the dominant negative Kir2.1 channel is Kir2.1AAA (SEQ. ID. NO. 6), in which the glycine, tyrosine, and glycine residues at positions 144-146 are substituted with alanine residues (see FIG. 5B for Kir2.1AAA amino acid sequence). The dominant negative Kir2.1AAA, in various embodiments, is a human dominant negative Kir2.1AAA channel.

In various embodiments, a dominant negative Kir2.1ER channel (SEQ. ID. NO. 7), having E138R and R148E mutations, is expressed in a cell to enhance hyperpolarization-activated cation inward current by disrupting potassium selectivity of the wild-type channel (see FIG. 5C for Kir2.1 ER amino acid sequence). As described herein, Kir2.1ER has been shown to function not only as a dominant negative Kir2.1 channel, but also as a hyperpolarization-activated cation channel.

Kir2.1 channels are multi-subunit channels, for which four monomeric subunits co-assemble following expression within a cell. Reference herein to a “Kir2.1 channel” includes reference to a subunit of a Kir2.1 channel.

An expression vector comprising DNA encoding functional HCN channel or a dominant negative Kir2.1 mutant channel may be made according to any known or future developed technique. In various embodiments, an expression vector includes DNA encoding a functional HCN channel and a dominant negative Kir2.1 channel. Of course, a functional HCN channel and a dominant negative Kir2.1 channel can be co-expressed by the same cell via different expression vectors. In some embodiments, whether or not included in the same expression vector, a functional HCN channel and a dominant negative Kir2.1AAA channel are co-expressed. In some embodiments, a functional HCN channel and a dominant negative Kir2.1ER channel are co-expressed. In some embodiments, a dominant negative Kir2.1AAA channel and a dominant negative Kir2.1ER channel are co-expressed.

The HCN channels or dominant negative Kir2.1 channel may be modified to form a chimeric molecule comprising HCN or Kir2.1 fused to another, heterologous polypeptide or amino acid sequence. In various embodiments, such a chimeric molecule includes a fusion of the HCN or dominant negative Kir2.1 channel with a tag polypeptide that provides an epitope to which an anti-tag antibody can selectively bind. The epitope tag is typically placed at the amino- or carboxyl-terminus of the channel. The presence of such epitope-tagged forms of the HCN or dominant negative Kir2.1 channels can be detected using an antibody against the tag polypeptide. Various suitable tag polypeptides and their respective antibodies are well known in the art, examples of which include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

DNA encoding an HCN channel or a Kir2.1 channel may be obtained from a cDNA library prepared from tissue believed to possess mRNA of the channel and to express it at a detectable level. Accordingly, human HCN channel or a Kir2.1 channel DNA can be conveniently obtained from a cDNA library prepared from human tissue.

Libraries can be screened with probes (such as antibodies to the HCN channel or the Kir2.1 channel or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding an HCN channel or a Kir2.1 channel is to use PCR methodology [Sambrook et al., supra; Dieffenbach et al., PCR Primer: A Laboratory Manual (Cold Spring Harbor Laboratory Press, 1995)].

When screening a cDNA library, the oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Nucleic acid having protein coding sequence may be obtained by screening selected cDNA or genomic libraries, and, if necessary, using conventional primer extension procedures as described in Sambrook et al., supra, to detect precursors and processing intermediates of mRNA that may not have been reverse-transcribed into cDNA.

While the above description is fairly generic, one skilled in the art will understand that the methods employed in the references associated with the Genbank Accession Numbers provided herein or otherwise obtainable may be used or routinely modified to obtain DNA or RNA encoding an HCN channel or a Kir2.1 channel.

Host cells may be transfected or transformed with expression or cloning vectors described herein for HCN or Kir2.1 production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to those of ordinarily skill in the art and include, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. For mammalian cells that do not contain cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning the DNA in the vectors herein include prokaryote, yeast, or higher eukaryote cells. Suitable prokaryotes include eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. Alternatively or in addition, in vitro methods of cloning, e.g., PCR or other nucleic acid polymerase reactions are suitable.

Suitable host cells for the expression of glycosylated HCN or Kir2.1 channels include those derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFP (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is within the routine ability skill in the art.

In various embodiments, myocardial cells, such as cardiomyoctes, Purkinje cells, or the like are used as the host cells. Cardiac stem cells, either derived from adult hearts, or from fetal or embryonic tissue, could be used as autologeous or allogeneic cells. Such cells would endogenously express a Kir2.1 channel. However, cells that do not endogenously express such a channel may be employed as well. In such cells, an exogenous wild-type or otherwise functional Kir2.1 channel may be co-expressed with a dominant negative Kir2.1 channel according to any known or future developed technique, including those described herein. Such cells include but are not limited to fibroblasts (cardiac or other origin), mesenchymal stem cells, and bone marrow derived stem cells. Preferably such cells would be able to form gap junctions with the host cardiac tissue.

The nucleic acid (e.g., cDNA or genomic DNA) encoding an HCN channel or Kir2.1 channel, whether dominant negative or functional, may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to those of skill in the art.

The HCN channel or Kir2.1 channel may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the HCN channel- or Kir2.1 channel-encoding DNA that is inserted into the vector.

Expression vectors or cloning vectors may include a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter operably linked to the HCN channel- or Kir2.1 channel-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgamo (S.D.) sequence operably linked to the DNA encoding HCN or Kir2.1 channel.

HCN channel or Kir2.1 channel transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the HCN or Kir2.1 channel by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the HCN or Kir2.1 channel coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding a HCN or Kir2.1 channel.

For example, a viral vector, such as an adeno-associated viral (AAV) vector may be operatively linked components of control elements. For example, a typical vector includes a transcriptional initiation region, a nucleotide sequence of the protein to be expressed, and a transcriptional termination region. Typically, such an operatively linked construct will be flanked at its 5 and 3 regions with AAV ITR sequences, which are viral cis elements. The control sequences can often be provided from promoters derived from viruses such as, polyoma, Adenovirus 2, cytomegalovirus, and Simian Virus 40. Viral regulatory sequences can be chosen to achieve a high level of expression in a variety of cells. Alternatively, ubiquitously expressing promoters, such as the early cytomegalovirus promoter can be utilized to accomplish expression in any cell type. A third alternative is the use of promoters that drive tissue specific expression. This approach is particularly useful where expression of the desired protein in non-target tissue may have deleterious effects. Thus, according to various embodiments, the vector contains the proximal human brain natriuretic brain (hBNP) promoter that functions as a cardiac-specific promoter. For details on construction of such a vector see LaPointe et al., “Left Ventricular Targeting of Reporter Gene Expression In Vivo by Human BNP Promoter in an Adenoviral Vector,” Am. J. Physiol. Heart Circ. Physiol., 283:H1439-45 (2002).

Vectors may also contain cardiac enhancers to increase the expression of the transgene in the targeted myocardial cells. Such enhancer elements may include the cardiac specific enhancer elements derived from Csx/Nkx2.5 regulatory regions disclosed in the published U.S. Patent Application 20020022259, the teachings of which are herein incorporated by reference.

Introducing the AAV vector into a suitable host, such as yeast, bacteria, or mammalian cells, using methods well known in the art, can produce AAV viral particles carrying the sequence of choice.

Expression vectors containing DNA encoding a functional HCN channel or a dominant negative Kir2.1 channel may be administered in vivo in any known or future developed manner. In various embodiments, the expression vectors are packaged into viruses, such as adenoviruses, and are delivered in proximity to targeted cells, tissue or organs. In various embodiments, the expression vectors are packaged into adenoviruses, such as helper-dependent adeno viral vector (HDAd) or adeno-associated virus pseudo-type 9 (AAV2/9). HDAd virus packaging typically illicits less of an immunogenic response in vivo compared to some other adenoviruses and thus allows for longer term expression. AAV2/9 packaging can result in cardiac tropism as well as a prolonged expression time frame. Other viruses of clinical relevance include lentiviruses. Replication deficient lentiviruses are RNA viruses, which can integrate into the genome and lead to long-term functional expression. Their capacity is up to 8 kb, which is an advantage over AAV, however, they have a relatively fragile envelope. Lentiviral vectors, AAV vectors, and HD AdV all are relevant vector platforms for the delivery of the polynucleotides described in this invention.

Once the expression vectors are packaged into viruses, the viral vectors may then be administered to cardiac cells, such as cardiomyocytes, Purkinje cells, or conductive tissue, SAN or AVN, cardiac fibroblasts, or generally to the heart or portions thereof.

Alternatively, non-viral delivery systems are employed. For example, liposomes, DNA complexes, plasmis, liposome complexes, naked DNA, DNA-coated particles, or polymer based systems may be used to deliver the desired sequence to the cells. The above-mentioned delivery systems and protocols therefore can be found in Gene Targeting Protocols, Kmeic 2ed., pages 1-35 (2002) and Gene Transfer and Expression Protocols, Vol. 7, Murray ed., Pages 81-89 (1991).

If it is desired to obtain biological pacemaker activity of a heart, or a portion thereof, a sufficient number of cells should express one or more transgene capable of enhancing hyperpolarization activated cation current and disrupting inwardly rectifying potassium current. As in the case of the real SA node, the effective number of HCN and Kir2.1 dominant negative expressing-cells to produce biological pacemaking is determined by space constant, the distance at which potential change from a source (a myocyte, for instance) decreases by 67% of the original value. In order to overcome the hyperpolarizing effect from ventricular or atrial myocytes, the leading pacemaker site should be several space constants away from the muscle tissues.

By way of example, for HDAd, a typical dose may range from 1×10⁵ to 1×10¹¹ infectious units (IU) per injection site due to the dependence fo transduction efficiency on the host tissue and specific region of the tissue. By way of another example, a typical rAAV2/9 dose may range from 1×10¹⁰ to 1×10¹⁴.

Delivery of an expression vector to a heart can be carried out according to any method known or developed in the art. To generate a biological pacemaker, the expression vector may reach only a small portion of targeted cells in an area of liminal dimension (i.e., about 0.5-1.0 mm across). The expression vector may be injected directly into the myocardium as described by R. J. Guzman et al., Circ. Res., 73:1202-1207 (1993). The delivery process may further include increasing microvascular permeability using routine procedures, including delivering at least one permeability agent prior to or during delivery of the expression vector. Infusion volumes in the range of about 10 uL to 100 mL are typically useful. Methods for targeting non-viral vector expression constructs to solid organs, for example, the heart, have been developed such as those described in U.S. Pat. No. 6,376,471, the teachings of which are hereby incorporated herein by reference to the extent that they do not conflict with the disclosure presented herein.

Therapeutic methods may include delivery of an effective amount of an expression vector to myocardial cells, such as cardiac atrial cells, Purkinje fiber cells or ventricular cells, to increase the intrinsic pacing rate of these cells to resemble the pacing rate of the SA node cells. The delivery or administration may be accomplished by injection, catheter and other delivering vehicle known or developed in the art. A delivery system for delivering genetic material in a targeted area of the heart is described in PCT Publication No. WO 98/02150, the teachings of which are herein incorporated by reference tp the extent that to does not conflict with the disclosure presented herein.

An expression vector including a polynucleotide encoding an HCN channel or a dominant negative Kir2.1 channel can be delivered into a cell by, for example, transfection or transduction procedures. Transfection and transduction refer to the acquisition by a cell of new genetic material by incorporation of added nucleic acid molecules. Transfection can occur by physical or chemical methods. Many transfection techniques are known to those of ordinary skill in the art including, without limitation, calcium phosphate DNA co-precipitation, DEAE-dextrin DNA transfection, electroporation, naked plasmid adsorption, and cationic liposome-mediated transfection. Transduction refers to the process of transferring nucleic acid into a cell using a DNA or RNA virus. Suitable viral vectors for use as transducing agents include, but are not limited to, retroviral vectors, lentiviral vectors, adenoviral vectors, adeno associated viral vectors, vaccinia viral vectors, and Semliki Foret viral vectors.

When it is desired to titrate the expression of two or more exogenous channels relative to each other (e.g. a HCN channel and a dominant negative Kir2.1 channel), differential expression of the channels can be accomplished by generating vectors with varied promoters or administration of differing dosages. Alternatively, multiple transgenes can be co-delivered by compound vectors as is known by those skilled in the art.

Expression vectors as described herein can be targeted to particular cells. For example, a receptor expressed on the surface of Purkinje cells is the cysteinyl leukotriene 2 receptor (CysLT₂). This receptor distinguishes Purkinje cells from neighboring cells such as ventricular cells and can be utilized to target expression vectors preferentially to Purkinje cells. However, it is to be understood that any receptor specific to Purkinje cells may be utilized for specific targeting.

Targeted delivery may entail modification of the vehicle delivering the construct. Several methods for modification of viral vectors are possible. For example, viral protein capsids or proteins of the viral envelope may be biotinylated for subsequent coupling to a biotinylated antibody directed against a specific receptor or ligand therefore via a streptavidin bridge.

Alternatively, the viral delivery vehicle may be genetically modified so that it expresses a protein ligand for a specific receptor. The gene for the ligand is introduced within the coding sequence of a viral surface protein by for example, insertional mutagenesis, such that a fusion protein including the ligand is expressed on the surface of the virus. For details on this technique see Han et al., “Ligand-Directed Retroviral Targeting of Human Breast Cancer Cells,” Proc. Natl. Acad. Sci., 92:9747-9751 (1995). Viral delivery vehicles may also be genetically modified to express fusion proteins displaying, at a minimum, the antigen-binding site of an antibody directed against the target receptor. See e.g., Jiang et al., “Cell-Type-Specific Gene Transfer into Human Cells with Retroviral Vectors That Display Single-Chain Antibodies,” J. Virol., 72:10148-10156 (1998).

Construct delivery vehicles may also be targeted to specific cells types utilizing bispecific antibodies produced by the fusion of anti-viral antibody with anti-target cell antibody. For details on this technique see Haisma et al., “Targeting of Adenoviral Vectors Through a Bispecific Single-Chain Antibody,” Cancer Gene Ther., 7:901-904 (2000) and Watkins et al., “The Adenobody Approach to Viral Targeting: Specific and Enhanced Adenoviral Gene Delivery,” Gene Ther., 4:1004-1012 (1997).

Targeted construct delivery provides numerous advantages including increased transduction efficiency and the avoidance of genetic modification of untargeted cells. Any technique for targeted gene therapy may be employed to target the construct of the invention to cells of interest.

As will be appreciated by those skilled in the art, the genetic manipulations described here may be practiced on stem cells without departing from the scope of the invention. The genetically modified stem cells can then be administered to the desired myocardial cells to elicit pacemaking activity or suppress conduction characteristics. For example, cardiac myocardial cells derived from stem cells may be treated with the genetic procedures described herein and implanted into a heart (e.g. ventricular muscle) with a catheter or by direct epicardial injection into the ventricular tissue.

In various embodiments, hyperpolarization-activated cation conductance of a cell is enhanced and inward rectifying potassium conductance is disrupted. The cell may originally be a non-pacemaker cell, i.e. a cell that does not undergo spontaneous oscillating action potentials. In various embodiments, the non-pacemaker cell is transformed to a biological pacemaker cell by the expression of one or more exogenous polynucleotides that enhance hyperpolarization-activated cation conductance and disrupt inward rectifying potassium conductance. In various embodiments, the non-pacemaker cell is made to behave more like a SA node cell.

Any suitable technique for determining whether a hyperpolarization-activated cation conductance is enhanced or whether inward rectifying potassium conductance is disrupted may be employed. For example, whole cell patch clamp techniques and multi-electrode array tests with cardiac cells, such as, for example, human ES derived, mouse HL05, rat neonatal, and others, may be employed.

In the context of a heart, in vivo or ex vivo, any conventional or developed methods for detecting modulation of the cells of the heart by electrophysiological assay may be used to determine the cardiac action potential characteristics, such as action potential duration (APD). An example of such a method related to performing such tests is disclosed by Josephson M E, Clinical Cardiac Electrophysiology: Techniques and Interpretations, Lea & Febiger. (1993), pp 22-70, the teachings of which are hereby incorporated herein by reference to the extent they do not conflict with the present disclosure. Additionally or alternatively, modulation of cardiac electrical properties may be observed by performing a conventional electrocardiogram (ECG) before and after administration of the expression vector and inspecting the ECG results. ECG patterns from a heart's electrical excitation have been well studied. Various methods are known for analyzing ECG records to measure changes in the electrical potential in the heart associated with the spread of depolarization and repolarization through the heart muscle.

Employing such techniques, it will be readily identifiable to one skilled in the art as to whether a non-pacemaker cell is made to behave more like a SA node cell. For purposes of guidance, a brief discussion of some relevant characteristics of SA node cells follows. SA nodal cells do not have a stable resting potential and instead begin to spontaneously depolarize when their membrane potential reaches about −50 mV. Cells, such as SA nodal cells, that do not have a stable resting transmembrane potential, but instead increase spontaneously to the threshold value, causing regenerative, repetitive depolarization, are said to have automacity. The cells in the SA node are unique because not only do they have automacity, but also their firing rate is the highest among all cardiac cells that demonstrate automacity (e.g., AV node and Purkinje cells). The SA node's unique cells include a combination of ion channels that endow it with its automacity. A review of the features of cardiac electrical function and description of the current understanding of the ionic and molecular basis, thereof, can be found in Schram et al., “Differential Distribution of Cardiac Ion Channel Expression as a Basis for Regional Specialization in Electrical Function,” Circ. Res., Vol. 90, pages 939-950 (2002), the teachings of which are hereby incorporated herein by reference to the extent that they do not conflict with the present disclosure.

SA node cells do not have a stable resting potential primarily because of the lack of the I_{K1} and generally begin to depolarize immediately after the repolarization phase is complete. The maximum diastolic potential for SA node cells is approximately −50 mV compared to −78 mV and −85 mV for atrial and ventricular cells, respectively. The slow depolarization phase is mediated by activation of “funny current” (I_{f}), which is mediated at least in part by HCN cells, and T-type Ca²⁺ channels and deactivation of slow and rapid potassium (I_{Ks} and I_{Kr}, respectively). Recent studies suggest that the HCN4 isoform is the predominant subunit encoding for the cardiac funny current channel in the SA node. (See, e.g., “Molecular Characterization of the Hyperpolarization-activated Cation Channel in Rabbit Heart Sinoatrial Node,” J. Biol. Chem. 274:12835-12839 (1999)). The rate of pacemaker discharge in the SA node in a normally functioning heart is approximately in the range of about 60 to 100 beats per minute.

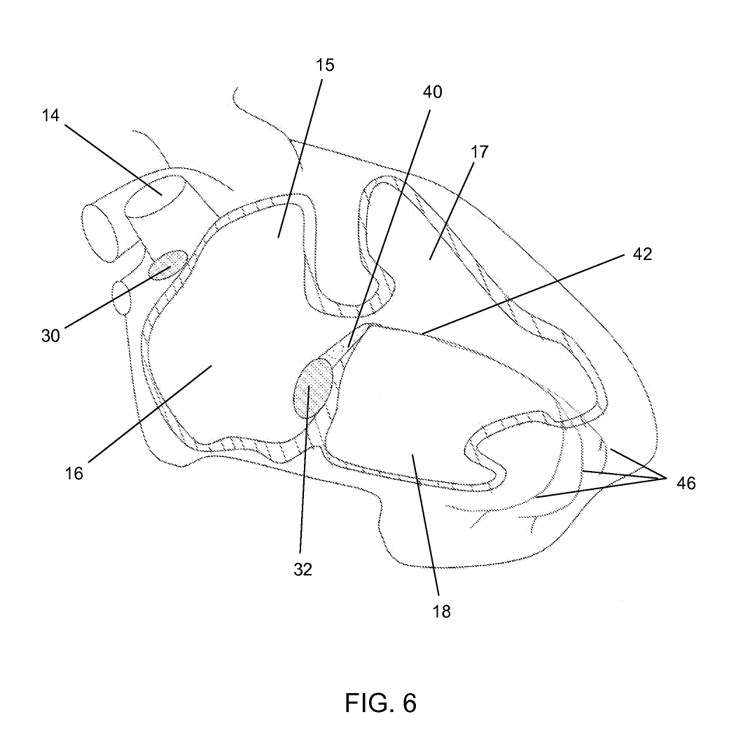

Referring now to FIG. 6, a schematic diagram is shown of a right side of a heart having an anterior-lateral wall peeled back to expose a portion of a heart's intrinsic conduction system and chambers of a right atrium 16 and a right ventricle (“RV”) 18. Pertinent elements of the heart's intrinsic conduction system, illustrated, in FIG. 6, include a SA node 30, an AV node 32, a bundle of His 40, a right bundle branch 42, and Purkinje fibers 46. SA node 30 is shown at a junction between a superior vena cava 14 and right atrium (“RA”) 16. An electrical impulse initiated at SA node 30 travels rapidly through RA 16 and a left atrium (not shown) to AV node 32. At AV node 32, the impulse slows to create a delay before passing on through a bundle of His 40, which branches, in an interventricular septum 17, into a right bundle branch 42 and a left bundle branch (not shown) and then, apically, into Purkinje fibers 46. Following the delay, the impulse travels rapidly throughout RV 18 and a left ventricle (not shown). Such flow of the electrical impulse creates an orderly sequence of atrial and ventricular contraction to efficiently pump blood through the heart. When a portion of the heart's intrinsic conduction system becomes dysfunctional, efficient pumping is compromised.

In various embodiments, an expression vector containing DNA encoding a functional DNA channel or a dominant negative Kir2.1 channel is introduced to cells of the RA 16. When the subject, which is typically a mammal, has a dysfunctional SA node 30 and does not suffer from atrial fibrillation and has an intact AV node 32, it may be desirable to introduce the expression vector to cells of the atrial appendage 15. However, if a subject has a non-functional AV node 32, it may be desirable to introduce the expression vector to cells downstream of the AV node, such as, for example, cells of the RV apex or LV epicardium.

In various embodiments, the expression vector is introduced to cells at the bundle of His 40.

In various embodiments, cells of the AV node are ablated. Such ablation is intended to prevent or reduce conduction of atrial impulses that may give rise to irregular ventricular rhythms. For example, such ablation may render ablated cells of the AV node incapable of transmitting rapid atrial activity during atrial fibrillation to the ventricles and a concurrent modification of other myocardial cells, such as ventricular cells or Purkinje fiber cells, into SA node-like pacemaker cells. In situations where the heart rate generated by these substitute intrinsic pacemakers is not sufficient to support normal systemic circulation, a pacemaker device with appropriate firing rate may be implemented.

When the AV node is ablated, it is desirable to generate a biological pacemaker in cardiac structures that are below the AV node in the cardiac conduction system. Potential sites for delivery of an expression vector containing DNA encoding a HCN channel or a dominant negative Kir2.1 channel include the bundle of His, major bundle branches, Purkinje fibers, or the ventricular muscle itself. These sites may be in either the left or right ventricle. In various embodiments, for ease of delivery of the expression vector (or carrier thereof) to the ventricles, the expression vector is delivered to the RV apex or left ventricle epicardium.

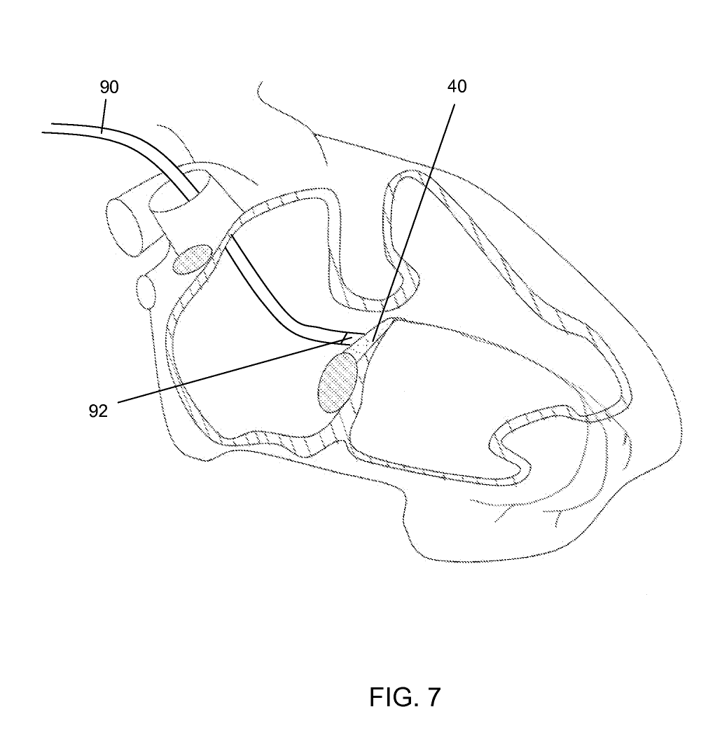

Referring now to FIG. 7, a schematic diagram is shown of the right side of a heart similar to that shown in FIG. 6, wherein a guide catheter 90 is positioned for delivery of the expression vector or carrier thereof (e.g., a virus). A venous access site (not shown) for catheter 90 may be in a cephalic or subclavian vein and means used for venous access are well known in the art, including the Seldinger technique performed with a standard percutaneous introducer kit. Guide catheter 90 includes a lumen (not shown) extending from a proximal end (not shown) to a distal end 92 that slideably receives a delivery system. Guide catheter 90 may have an outer diameter between approximately 0.115 inches and 0.170 inches and is of a construction well known in the art. Distal end 92 of guide catheter 80 may include an electrode (not shown) for mapping electrical activity in order to direct distal end 92 to an implant site near bundle of His 40 or other desirable location. A separate mapping catheter (not shown) may be used within lumen of guide catheter 90 to direct distal end 92 to an implant site near bundle of His 40 or other desirable location, a method well known in the art.

Myocardial cells may be modified to maximize the transformation of these cells into the primary pacemaker and to increase their intrinsic pacing rate to a level resembling that of the SA node. In various embodiments, the intrinsic pacing rate of the modified cells is increased to a level more closely resembling, preferably substantially identical to, that of the SA node. For example, the pacing rate of the modified cells may be increased to a level of at least about 85%, at least about 90%, or at least about 95% of the pacing rate of the SA node cells for a particular subject, such as a human patient, when the heart is functioning normally.

The pacing rate of any cardiac cell type is the product of the composition of channels expressed by the cell as well as electrotonic influences exerted by neighboring cells. For example, evidence suggests that the ventricles exert electrotonic influences on the Purkinje cells at the Purkinje-ventricular junction, thereby inhibiting its pacing rate. Thus, to be effective, proposed genetic modifications must take into account the wild type channel expression as well as influences exerted by neighboring cells.

The electrotonic influences of the ventricles can be decreased by partial electrical uncoupling of Purkinje fibers from the neighboring ventricular cells. Since electrical impulse spread through the ventricles via gap junctions, uncoupling the gap junctions in the vicinity of the genetic modification can help to decrease the electronic load on the Purkinje cell derived bio-pacemaker and enhance its pacing rate. Such a modification is particularly useful where the genetic modifications are performed in the more distal portions of the Purkinje fibers that are embedded in the ventricular endocardium.

Gap junctions can be uncoupled by interfering with the formation of connexons. Ventricular gap junctions can be preferentially uncoupled while leaving the gap junctions of the Purkinje cells or other cells intact, by the targeted interference of connexin 43 (CX43), the predominant form of connexin protein in the ventricular gap junctions.

Reference is now made to the following non-limiting examples. It will be apparent to those skilled in the art that many changes can be made in the embodiments described in the examples without departing from the scope of the present invention.

It will be understood to one of skill in the art that discussion in certain areas of the present disclosure is applicable to other areas of the disclosure. For example, one skilled in the art will readily recognize that the techniques described for modifying Kir2.1 may be applied to modification of HCN. By way of additional example, the discussion of species origin of cells and channels regarding HCN is applicable to dominant negative Kir2.1 channels, including Kir2.1ER.

### EXAMPLES

### Example 1

### Generation of Biological Pacemaker by Kir2.1 Gene Transfer

Efforts to engineer biological pacemakers have focused either on over-expression of HCN channels or suppression of I_{K1} to liberate endogenous pacemaker activity. Here we report a novel strategy designed to convert I_{K1} into a cationic nonselective “leak” current. We utilized Kir2.1 channel mutations (E138R and R148E, Kir2.1ER) which have been shown to render the channel non-selective, conducting Na+ as well as K+.

Co-expression of wild-type and the mutant channels by Ad-Kir2.1ER-IRES-Kir2.1WT in HEK293 cells yielded Ba2+-sensitive hyperpolarization-activated inward currents (−5.5 pA/pF at −80 mV) with a Vrev of −35.1±2.1 mV (n=5). The data demonstrates that over-expression of Kir2.1ER destabilizes the resting membrane potential established by Kir2.1 WT channels.

Equipped with this data, adenovirus expressing Kir2.1ER channels (Ad-Kir2.1ER-IRES-GFP) were expressed by a direct injection into the apex of guinea pig hearts. Ad-Kir2.1ER-IRES-GFP transduced cardiomyocytes exhibited spontaneous action potential oscillations (n=10) which was not observed in cardiomyocytes transduced with the reporter gene alone (Ad-GFP). Electrocardiograms (ECGs) performed three days after Ad-Kir2.1ER-IRES-GFP injection into guinea pig hearts showed idioventricular rhythms (n=6), while no such rhythms were seen in control animals (Ad-GFP, n=6).

Taken together, these results demonstrate that Kir2.1ER over-expression produces in vivo biological pacemaker activity through hyper-polarization activated, cation non-selective inward current. Thus, the present approach may combine the effect of HCN-overexpression and Kir2.1 dominant-negative strategy with a single Kir2.1ER gene expression in creating a biological pacemaker.

Materials and Methods

Construction of Ad-Kir2.1ER-IRES-Kir2.1WT: Briefly, BamHI and XbaI sites were used to subclone the gene of interested after the internal ribosomal entry site (IRES) of an adenovirus shuttle vector pAdCMV-GFP—IRES. Adenoviruses were generated by Cre-lox recombination of purified psi 5 viral DNA and shuttle vector DNA in Cre4 cells. The recombinant viruses are plaque-isolated, amplified, and purified by CsCl gradients.

Co-expression of wild-type and Kir2.1ER channels in HEK293 cells: HEK293 cells were transfected with Ad-Kir2.1ER-IRES-Kir2.1WT as follows. The Kir2.1ER mutant and WT channels were cloned into one DNA construct with IRES in between. IRES allows the transcription and translation of the second gene without another promoter.

Ba²⁺-sensitive hyperpolarization-activated inward currents: Hyperpolarization-activated inward currents of Ad-Kir2.1ER-IRES-Kir2.1WT transfected HEK293 cells were obtained as follow. Briefly, normal Tyrode's solution containing 1 mM BaCl2 was washed in to block wild-type Kir2.1-encoded inward currents. The surviving current is analyzed as the hyperpolarization-activated inward currents.

Results

Co-expression of wild-type and Kir2.1ER channels by Ad-Kir2.1ER-IRES-Kir2.1WT in HEK293 cells yielded Ba²⁺-sensitive hyperpolarization-activated inward currents as show in FIG. 8.

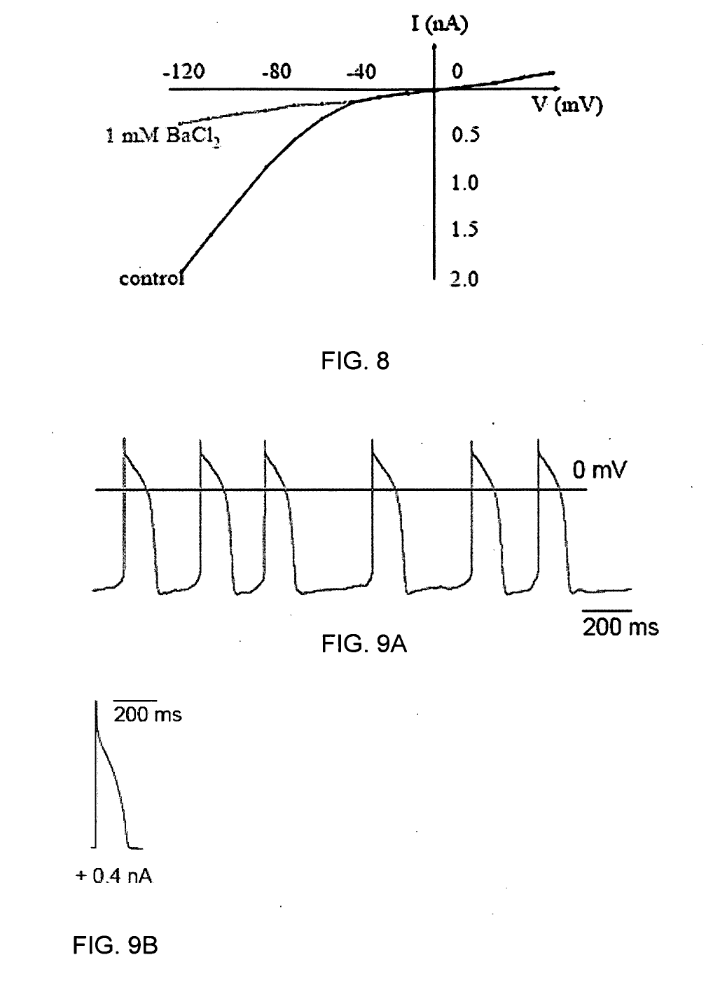

Cardiomyocytes in vivo transduced with Ad-Kir2.1ER-IRES-GFP displayed spontaneously oscillating APs (FIG. 9A). Control myocytes transduced with GFP alone generated an AP only after 4 ms external stimulus with 0.2-0.6 nA of depolarizing current (FIG. 9B).

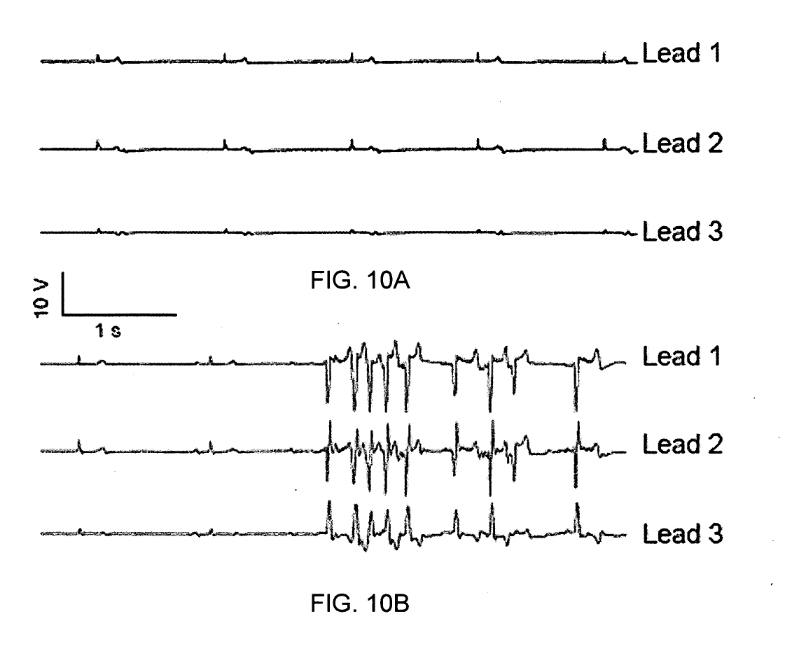

ECGs recorded three days after Ad-Kir2.1ER-IRES-GFP injection into the apex of a guinea pig heart showed idioventricular rhythms (n=6, FIG. 10A), while no such rhythms were seen in control animals (Ad-GFP, n=6, FIG. 10B). Intrinsic heart rates of the guinea pigs were reduced with i.p. injection of methacholine.

### Example 2

### Generation of Viral Vectors for Expression of Kir2.1AAA and HCN

Plasmids, Viruses and Cell Lines

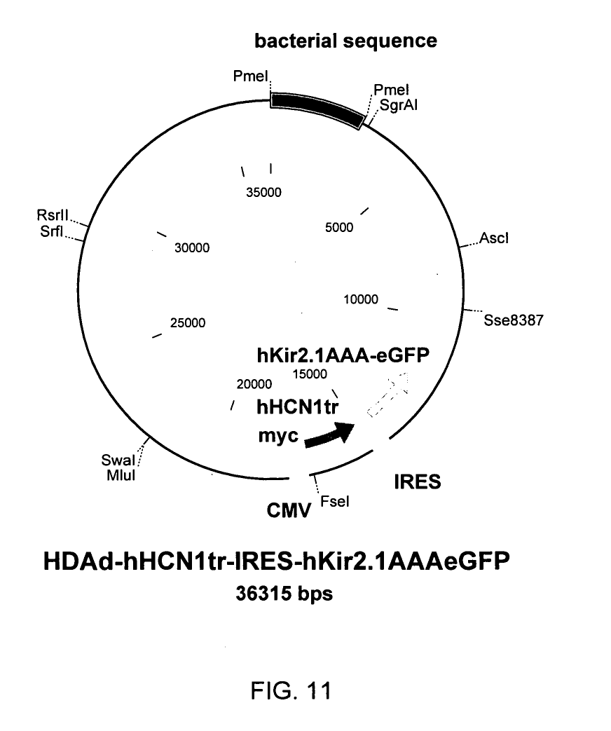

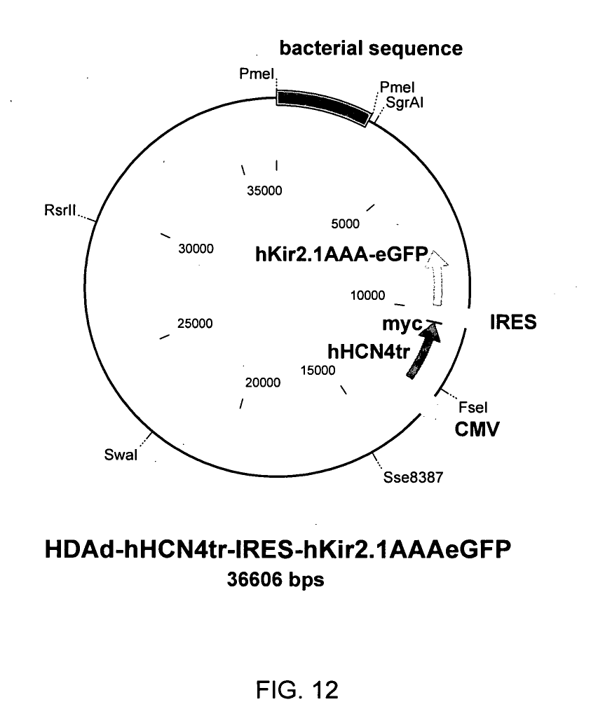

HD adenovirus vector system was provided by Microbix. HDAd-HCN1tr-IRES-Kir2.1AAAeGFP was constructed by placing a linker containing a NotI site into the AseI site of pHCN1tr-IRES-Kir2.1AAAeGFP. The resulting plasmid (see FIG. 11) was cloned into the NotI site of pC4HSU, a plasmid containing the HD adenovirus backbone and stuffer DNA. Expression of Kir2.1AAA should result in an amino acid of SEQ. ID. NO. 6, see FIG. 5B. Expression of HCN1tr should result in an amino acid comprising amino acids 1-581 of SEQ. ID. NO. 1 with amino acids 582-890 truncated (SEQ. ID. NO. 8), see FIG. 1B.

To construct HDAd-HCN4tr-IRES-Kir2.1AAAeGFP, a linker containing AscI sites was cloned into pHCN4tr-IRES-Kir2.1AAAeGFP. The AscI fragment from pHCN4tr-IRES-Kir2.1AAAeGFP containing the entire transgene was then cloned into the homologous site in pC4HSU. Expression of Kir2.1AAA should result in an amino acid of SEQ. ID. NO. 6, see FIG. 5B. Expression of HCN4tr should result in an amino acid comprising amino acids 1-710 of SEQ. ID. NO. 1 with amino acids 711-1203 truncated (SEQ ID NO. 9), see FIG. 4B.

Helper virus 14 contains a modified packaging signal flanked by two loxP sites, and 2902 bp of human DNA, as described (Sandig et al.).

293Cre4 cells were maintained as previously described (Chen et al.).

It will be understood that HDAd-Kir2.1AAA-IRES-HCN1tr and HDAd-Kir2.1AAA-IRES-HCN4tr may be constructed according to the techniques described above or using other similar techniques.

HD Vector Rescue and Purification

HD vector production was carried out in 293Cre4 cells expressing Cre-recombinase (Microbix). Cells were transfected in six-well plates with 10 ug of PmeI-digested HDAd-HCN1tr-IRES-Kir2.1AAAeGFP or HDAd-HCN4tr-IRES-Kir2.1AAAeGFP using the Lipofectamine 2000 reagent (Invitrogen). Twelve hours after transfection, 293Cre4 cells were infected with H14 helper virus at a multiplicity of infection (m.o.i.)=5. After 48 hours, cells were lysed by three freeze/thaw cycles, and 6 ml of the lysate were used to infect a 15-cm dish of 293 Cre4 cells at ˜90% confluency together with helper virus. HDAd-HCN1tr-IRES-Kir2.1AAAeGFP or HDAd-HCN4tr-IRES-Kir2.1AAAeGFP was amplified by serial coinfections of 15-cm dishes of 293Cre4 cells with 10-50% of the crude lysate from the previous passage and H14 helper virus at an m.o.i. of 1 PFU/cell for serial passages 1-8 with increasing numbers of cells. At passage 8, HD vector from 100 15-cm dishes was purified by double CsCl banding.

### Example 3

### Prophetic Example

Adult guinea pigs may be infected by intramuscular injection (via catheter) of a solution of saline with a viral concentration range of approximately 3×10¹⁰ to 3×10¹⁴ plaque forming units (PFU) HDAd or AAV 2/9. The HDAd or AAV 2/9 may contain an expression vector having DNA encoding an HCN1 channel and a dominant negative Kir2.1 channel. The expression vector may contain a reporter gene, such as green fluorescence protein. DNA encoding a short polypeptide protein, such as myc-tag, which can serve as an antigen for verification of expression, may be inserted such that it will be expresses at the N- or C-terminal of the HCN1 channel or the Kir2.1 channel.

For targeted injection to the right side of the heart, the catheter may be guided to the right atrium, either via the superior vena cava or inferior vena cava, which by itself may be accessed via one of the femoral veins. The right ventricle may then be accessed by guiding the catheter through the tricuspid valve to target the left side of the heart, left atrium may be accessed from the right atrium via the septum primum. From the left atrium, the catheter may be guided through the bicuspid valve to the left ventricle. The AV node may be targeted by direct perfusion into the AV nodal branch of the right coronary artery. The left ventricle epicardium may be targeted via the coronary sinus.

Immunocytochemistry and immunohistochemistry may be used to verify or quantify expression levels of the exogenous channels. In vivo functional tests, such as ECGs, may be used to determine the effects of expression of the exogenous channels. Functional biopacemakers are expected to reveal ectopic beats on the lead II of an ECG recording with opposite polarity in the sinus rhythm.

Thus, embodiments of GENERATION OF BIOLOGICAL PACEMAKER ACTIVITY are disclosed. One skilled in the art will appreciate that the present invention can be practiced with embodiments other than those disclosed. The disclosed embodiments are presented for purposes of illustration and not limitation.

What is claimed is:

1. A method for generating a cell capable of undergoing spontaneously oscillating action potentials, the method comprising:
expressing an exogenous dominant negative Kir2.1 mutant inwardly rectifying potassium channel in the cell; and
expressing an exogenous hyperpolarization-activated cation (HCN) channel in the cell;
wherein the expression of the dominant negative Kir2.1 mutant and the HCN channel results in spontaneous oscillating action potentials in the cell.

2. The method of claim 1, wherein the cell is a cardiomyocyte.

3. The method of claim 1, wherein the Kir2.1 mutant is a Kir2.1AAA mutant.

4. The method of claim 1, wherein the Kir2.1AAA mutant has an amino acid sequence of SEQ ID NO: 6.

5. The method of claim 1, wherein the HCN channel is truncated.

6. The method of claim 1, wherein the HCN channel is a HCN1 channel.

7. The method of claim 6, wherein the HCN1 channel is truncated and has an amino acid sequence of SEQ ID NO: 8.

8. The method of claim 1, wherein the HCN channel is a HCN4 channel.

9. The method of claim 8, wherein the HCN4 channel is truncated and has an amino acid sequence of SEQ ID NO: 9.

10. The method of claim 1, wherein the cell is in a heart of an animal.

11. The method of claim 10, further comprising contacting the cell with a viral vector comprising first and second polynucleotides, wherein the cell is capable of expressing the Kir2.1 mutant from first polynucleotide, and wherein the cell is capable of expressing the HCN channel from the second polynucleotide.

12. The method of claim 11, wherein the viral vector is derived from an adenovirus.

13. The method of claim 11, wherein the viral vector is an HD adenovirus.

14. A method comprising:
identifying a cell that endogenously expresses an inwardly rectifying potassium channel; and
introducing into the cell a genetic construct comprising a polynucleotide that when expressed by the cell (i) disrupts the inwardly rectifying potassium current and (ii) increases inward hyperpolarization activated cation current.

15. The method of claim 12, wherein the polynucleotide encodes (i) a dominant negative Kir2.1 mutant and (ii) a hyperpolarization activated cation (HCN) channel.

16. The method of claim 14, wherein the dominant negative Kir2.1 mutant is a Kir2.1AAA mutant.

17. The method of claim 14, further comprising packaging the genetic construct into a viral vector, and wherein introducing the genetic construct into the cell comprises contacting the cell with the viral vector.

18. The method of claim 17, wherein the viral vector is a derivative of an adenovirus.

19. The method of claim 14, wherein the cell is a cardiomyocyte.

20. The method of claim 14, wherein the cell is in a heart of and animal.

21. An expression vector comprising:
a polynucleotide encoding a Kir2.1AAA channel; and
a polynucleotide encoding a HCN channel.

22. The expression vector of claim 21, wherein the HCN channel is an HCN1 channel or an HCN2 channel.

23. The expression vector of claim 21, wherein the HCN channel is truncated at a position following the channel's cyclic nucleotide binding site.

24. A cell comprising the expression vector of claim 21.


SEQUENCE LISTING (9 sequences)

SEQ ID NO: 1 - protein, 890 aa, Homo sapiens
MEGGGKPNSSSNSRDDGNSVFPAKASATGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIAPINYPQMTTLNSTSSTTTPTSRMRTQSPPVYTATSLSHSNLHSPSPSTQTPQPSAILSPCSYTTAVCSPPVQSPLAARTFHYASPTASQLSLMQQQPQQQVQQSQPPQTQPQQPSPQPQTPGSSTPKNEVHKSTQALHNTNLTREVRPLSASQPSLPHEVSTLISRPHPTVGESLASIPQPVTAVPGTGLQAGGRSTVPQRVTLFRQMSSGAIPPNRGVPPAPPPPAAALPRESSSVLNTDPDAEKPRFASNL

SEQ ID NO: 2 - protein, 889 aa, Homo sapiens
MDARGGGGRPGESPGATPAPGPPPPPPPAPPQQQPPPPPPPAPPPGPGPAPPQHPPRAEALPPEAADEGGPRGRLRSRDSSCGRPGTPGAASTAKGSPNGECGRGEPQCSPAGPEGPARGPKVSFSCRGAASGPAPGPGPAEEAGSEEAGPAGEPRGSQASFMQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPRNCWVSINGMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLNSGVFNNQENAIIQEIVKYDREMVQQAELGQRVGLFPPPPPPPQVTSAIATLQQAAAMSFCPQVARPLVGPLALGSPRLVRRPPPGPAPAAASPGPPPPASPPGAPASPRAPRTSPYGGLPAAPLAGPALPARRLSRASRPLSASQPSLPHGAPGPAASTRPASSSTPRLGPTPAARAAAPSPDRRDSASPGAAGGLDPQDSARSRLSSNL

SEQ ID NO: 3 - protein, 774 aa, Homo sapiens
MEAEQRPAAGASEGATPGLEAVPPVAPPPATAASGPIPKSGPEPKRRHLGTLLQPTVNKFSLRVFGSHKAVEIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPPDCWVSINHMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLLRIGKKNSILQRKRSEPSPGSSGGIMEQHLVQHDRDMARGVRGRAPSTGAQLSGKPVLWEPLVHAPLQAAAVTSNVAIALTHQRGPLPLSPDSPATLLARSAWRSAGSPASPLVPVRAGPWASTSRLPAPPARTLHASLSRAGRSQVSLLGPPPGGGGRRLGPRGRPLSASQPSLPQRATGDGSPGRKGSGSERLPPSGLLAKPPRTAQPPRPPVPEPATPRGLQLSANM

SEQ ID NO: 4 - protein, 1203 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 5 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 6 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIAAAFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 7 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIRTQTTIGYGFECVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 8 - protein, 581 aa, Homo sapiens
MEGGGKPNSSSNSRDDGNSVFPAKASATGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRR

SEQ ID NO: 9 - protein, 710 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDR